(19) United States
(12) Patent Application Publication
(10) Pub. No.: US 2025/0099610 A1
(43) Pub. Date: Mar. 27, 2025

(54) MAGNETITE-BASED MICRO/NANOROBOT AND PREPARATION METHOD AND USE THEREOF

(71) Applicant: CHINA UNIVERSITY OF GEOSCIENCES (WUHAN), Wuhan (CN)

(72) Inventors: Huaming YANG, Wuhan (CN); Ying FENG, Wuhan (CN)

(73) Assignee: CHINA UNIVERSITY OF GEOSCIENCES (WUHAN), Wuhan (CN)

(21) Appl. No.: 18/977,356

(22) Filed: Dec. 11, 2024

(30) Foreign Application Priority Data
Feb. 2, 2024 (CN) 202410158537.4

(51) Int. Cl. A61K 47/69; A61K 9/00; A61K 31/05; A61K 31/137; A61K 41/00; A61K 47/61

(52) U.S. Cl. CPC A61K 47/6957; A61K 9/0009; A61K 31/05; A61K 31/137; A61K 41/00; A61K 47/61

(57) ABSTRACT

A magnetite-based micro/nanorobot and a preparation method and use thereof are provided. The magnetite-based micro/nanorobot uses a polydopamine-coated magnetite as a carrier that is loaded with an anti-inflammatory drug and is finally coated with sodium alginate. The magnetite-based micro/nanorobot of the present disclosure improves a loading effect of the drug resveratrol and has an excellent anti-inflammatory effect. The magnetite-based micro/nanorobot of the present disclosure does not have obvious cytotoxicity, and exhibits excellent biocompatibility and high safety performance. The magnetite-based micro/nanorobot of the present disclosure allows the responsive release according to different pH values of a gastrointestinal environment. Compared with the traditional drug carriers, the magnetite-based micro/nanorobot of the present disclosure has a high rate and prominent targetability, and can be prepared by a method involving simple steps and easy operations.

### CROSS-REFERENCE TO RELATED APPLICATIONS

This application claims priority to Chinese Patent Application No. 202410158537.4 with a filing date of Feb. 2, 2024. The content of the aforementioned application, including any intervening amendments thereto, is incorporated herein by reference.

### TECHNICAL FIELD

The present disclosure relates to the technical field of drug carriers, and in particular to a magnetite-based micro/nano robot and a preparation method and use thereof.

### BACKGROUND

Inflammatory bowel disease is a refractory and recurrent gastrointestinal disease. The current clinical strategies for treating inflammatory bowel disease are based on the frequent and high-dose administration of a drug such as the aminosalicylic acid preparation, the antibiotic, the corticosteroid, and the immunosuppressant to ensure a high concentration of the drug at the inflammation site. However, it is difficult for the conventional enteritidis-treating drugs to accurately reach the lesion and allow the accurate treatment relying on the passive transport of the body fluid, which can lead to a series of adverse reactions such as side effects of systemic drug absorption. Therefore, the accurate treatment of enteritis, namely, the accurate delivery and targeted release of the drug, is currently the key research content for inflammatory bowel disease.

Micro/nanorobots are miniaturized smart devices that can convert internal energy or an external physical stimulus into the mechanical force in the micro/nanoscale. Micro/nanorobots can actively shuttle through organisms under the driving of various propulsion modes to reach lesion regions where the traditional drug delivery modes are difficult to reach. The common driving modes include chemical driving, magnetic driving, photic driving, ultrasonic driving, or the like. The chemical driving, as the earliest driving mode investigated, has been widely used in the treatment of gastrointestinal diseases. Generally, the self-driving of a chemically-driven micro/nanorobot is allowed by gas produced from decomposition of a biological fluid as a substrate in a gastrointestinal tract. However, it is difficult to adjust a motion velocity and a direction of a chemically-driven micro/nanorobot. Thus, a chemically-driven micro/nanorobot cannot undergo a continuous and directional motion. Currently, the generation of a magnetic field externally can allow a variety of motion modes for a magnetically-driven micro/nanorobot to improve the navigability and controllability. The magnetic field can also induce a magnetically-driven micro/nanorobot to produce a high temperature as an effective means for treating diseases. However, the research on the design of a magnetically-driven micro/nanorobot for treating inflammatory bowel disease has not been reported.

### SUMMARY OF PRESENT INVENTION

An objective of the present disclosure is to provide a magnetite-based micro/nanorobot and a preparation method and use thereof in view of the above-mentioned deficiencies of the prior art.

A magnetite-based micro/nanorobot is provided, the magnetite-based micro/nanorobot uses a polydopamine-coated magnetite as a carrier that is loaded with an anti-inflammatory drug and is coated with sodium alginate. Further, the anti-inflammatory drug is resveratrol.

Further, the polydopamine-coated magnetite is produced through polymerization of dopamine hydrochloride on a surface of a magnetite under an alkaline condition.

Further, the resveratrol is adsorbed on a surface of the polydopamine-coated magnetite through an electrostatic interaction.

Further, the magnetite is spherical and has a nano-scale size; and/or, the magnetite-based micro/nano robot is spherical and has a nano-scale size.

A preparation method of the magnetite-based micro/nanorobot is provided, including the following steps:
S1, preparing the magnetite through hydrothermal synthesis;
S2, polymerizing the dopamine hydrochloride on the surface of the magnetite under the alkaline condition to obtain the carrier;
S3, dissolving and adding the anti-inflammatory drug of resveratrol to the carrier, shaking in the dark for a specified period of time, washing, and lyophilizing to obtain a magnetite complex loaded with the resveratrol;
S4, coating the sodium alginate on a surface of the magnetite complex loaded with the resveratrol through a water-in-oil emulsion process to obtain the magnetite-based micro/nanorobot.

Further, in the S3, the anti-inflammatory drug is resveratrol, and the resveratrol is added to methanol and shaken in the dark for thorough dissolution; and a concentration of the resveratrol dissolved is 2.5 mg/mL to 7.5 mg/mL.

Further, in the S3, a mass ratio of the carrier to the resveratrol is 1: (5-15).

Further, the S4 includes: adding the sodium alginate and the magnetite complex loaded with the resveratrol to deionized water, and mechanically stirring for thorough dissolution to obtain an aqueous phase; mixing liquid paraffin, Span-80, and Tween-80, stirring until clear, and cooling to obtain an oil phase; and adding the oil phase to the aqueous phase.

A use of the magnetite-based micro/nanorobot described above in preparation of a drug for inhibiting inflammatory cells and treating an inflammation is provided.

In the magnetite-based micro/nanorobot of the present disclosure, magnetite nanoparticles are adopted as a core, and a thin polydopamine nanoparticle layer is polymerized in situ on a surface of the magnetite nanoparticles, such that the surface is positively-charged and can electrostatically adsorb an anti-inflammatory principal drug, which effectively improves a drug-loading rate. Polydopamine has an effective oxidation-reduction ability and thus can clear reactive oxygen species generated at an inflammation site. Resveratrol can maintain the stability of intestinal floras. An anti-inflammatory ability of polydopamine can be effectively combined with a flora-regulating function of resveratrol to well play a therapeutic role.

In addition, the coating of sodium alginate makes the magnetite-based micro/nanorobot have pH responsiveness, which can allow the accurate release at an intestinal site and improve an efficiency of an inflammation treatment. Therefore, the prepared magnetite-based micro/nanorobot provides a brand-new idea for the intervention and treatment of inflammatory bowel disease.

Compared with the prior art, the technical solutions of the present disclosure have the following advantages:
(1) The magnetite-based micro/nanorobot of the present disclosure improves a loading effect of the drug resveratrol and has an excellent anti-inflammatory effect.
(2) The magnetite-based micro/nanorobot of the present disclosure does not have obvious cytotoxicity, and exhibits excellent biocompatibility and high safety performance.
(3) The magnetite-based micro/nano robot of the present disclosure allows the responsive release according to different pH values of a gastrointestinal environment.
(4) Compared with the traditional drug carriers, the magnetite-based micro/nano robot of the present disclosure has a high rate and prominent targetability, and can be prepared by a method involving simple steps and easy operations.
(5) The magnetite-based micro/nano robot of the present disclosure has oxidation resistance and can effectively clear free radicals generated at an inflammation site.
(6) The magnetite-based micro/nanorobot of the present disclosure can be heated up under an action of an external alternating magnetic field, and has a stable magnetocaloric effect and a potential of magnetocaloric therapy, which can accelerate the recovery of an inflammatory disease.

### BRIEF DESCRIPTION OF THE DRAWINGS

FIG. 1a and FIG. 1b show transmission electron microscopy (TEM) images of a magnetite-based micro/nanorobot according to Example 1 of the present disclosure;

FIG. 2 shows motion velocity analysis results of the magnetite-based micro/nanorobot according to Example 1 of the present disclosure;

FIG. 3 is a schematic diagram of cytotoxicity of the magnetite-based micro/nanorobot according to Example 1 of the present disclosure;

FIG. 4 shows drug release curves of the magnetite-based micro/nanorobot according to Example 1 under different pH conditions;

FIG. 5 shows temperature change curves of the magnetite-based micro/nanorobot according to Example 1 under actions of alternating magnetic fields;

FIG. 6 shows a magnetocaloric curve of the magnetite-based micro/nanorobot according to Example 1 under an action of an alternating magnetic field; and

FIG. 7 is a schematic diagram of oxidation resistance of the magnetite-based micro/nanorobot according to Example 1 and components thereof.

### DETAILED DESCRIPTION OF THE EMBODIMENTS

The technical solutions of the present disclosure are described in further detail below with reference to the specific embodiments and accompanying drawings, but the present disclosure is not limited thereto.

### Preparation of a Polydopamine-Coated Magnetite as a Carrier

A magnetite is prepared according to the following process: 1.35 g of FeCl₃·6H₂O and 40 mL of ethylene glycol are weighed and added to a reaction bottle, and 3.5 g of sodium acetate and 1.0 g of polyethylene glycol are weighed and added to the reaction bottle to obtain a first system. The first system is fully stirred at room temperature to allow complete dissolution and then added to a 50 mL polytetrafluoroethylene (PTFE) high-pressure reactor. The high-pressure reactor is sealed and placed in a blast drying oven to allow a reaction at 200° C. for 12 h to obtain a second system. The second system is cooled to room temperature and centrifuged at 8,500 rpm to obtain a precipitate. The precipitate is washed with absolute ethanol three times and then dried at 60° C. to obtain the magnetite, which is denoted as Fe₃O₄.

The polydopamine-coated magnetite is prepared according to the following process: 0.12 g of Tris is weighed and added to a beaker, 100 mL of deionized water is added to the beaker, and ultrasonic dispersion is conducted for 5 min to obtain a Tris solution. A pH of the Tris solution is adjusted with 1 M hydrochloric acid to 8.5 to obtain an alkaline buffer solution. Then the alkaline buffer solution is poured into a 250 mL round-bottomed flask, 100 mg of the Fe₃O₄ powder prepared above is added to the round-bottomed flask, and mechanical stirring is conducted for 20 min to allow full dispersion. 0.02 g of dopamine hydrochloride is weighed and added to the round-bottomed flask, and mechanical stirring is conducted at room temperature for 5 h to ensure complete polymerization to obtain a product. Finally, the product is collected with a magnet, washed with deionized water 3 times, and dispersed in deionized water for storage, which is denoted as Fe₃O₄@PDA.

### Preparation of a Resveratrol-Loaded Magnetite Complex

The resveratrol-loaded magnetite complex is prepared according to the following process: 25 mg of a resveratrol powder is weighed, added to 20 mL of methanol, and ultrasonically dispersed for 5 min to obtain a resveratrol solution. 2.5 mg of the Fe₃O₄@PDA prepared above is taken and added to the resveratrol solution, ultrasonic dispersion is conducted for 5 min to obtain a mixed solution, and the mixed solution is shaken at 37° C. in the dark for 24 h to obtain a product. Finally, the product is collected with a magnet, washed with methanol 3 times, and lyophilized to obtain the resveratrol-loaded magnetite complex, which is denoted as Fe₃O₄@PDA-Res.

### Preparation of a Magnetite-Based Micro/Nanorobot

### Example 1

(1) Preparation of an oil phase: 57.2 mL of liquid paraffin was added to a round-bottomed flask and heated to 50° C. in a water bath, 1.2 mL of Span-80 and 0.4 mL of Tween-80 were added to the round-bottomed flask to obtain a mixed solution, and the mixed solution was mechanically stirred until clear and then cooled to 37° C.
(2) Preparation of an aqueous phase: 12 mg of the Fe₃O₄@PDA-Res prepared above and 20 mg of sodium alginate were added to 20 mL of deionized water, and mechanical stirring was conducted for 30 min.
(3) The aqueous phase was added at a rate of 500 μL/min dropwise to the oil phase by a syringe pump, during which a temperature of 37° C. and a rotational speed of 700 rpm were maintained. After the oil phase and the aqueous phase were fully mixed, mechanical stirring was further conducted for 1 h to obtain a homogeneous and stable milky-white water-in-oil emulsion. A 5% calcium chloride solution was added dropwise to the water-in-oil emulsion to allow cross-linking, and mechanical stirring was conducted to allow curing for 2 h to obtain a product. The product was washed 3 times with each of isopropyl alcohol and deionized water and then lyophilized to obtain the magnetite-based micro/nano robot.

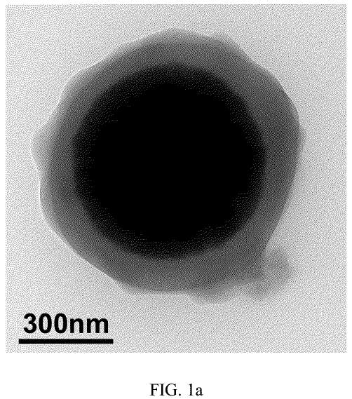

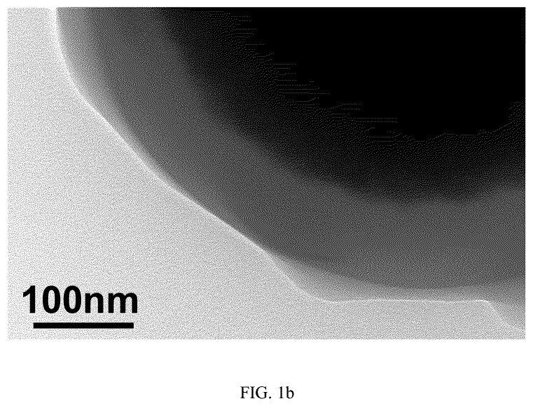

FIG. 1a and FIG. 1b show TEM images of the magnetite-based micro/nano robot in Example 1 of the present disclosure.

It can be seen from this figure that the magnetite-based micro/nano robot prepared in Example 1 has a spherical structure with a diameter of about 700 nm, and in the magnetite-based micro/nano robot, sodium alginate is uniformly coated on a surface of Fe₃O₄@PDA-Res. It is easy to control a path of the magnetite-based micro/nano robot with the spherical structure.

### Evaluation of a Motion Velocity of the Magnetite-Based Micro/Nanorobot

In order to objectively evaluate the motion performance of the magnetite-based micro/nanorobot prepared in Example 1, motion velocities of the magnetite-based micro/nanorobot under external 5 mT magnetic fields at different frequencies were tested in this example. Real-time motions of each micro/nanorobot at X and Y coordinates were recorded by the particle tracking software Video Spot Tracker, and according to the data, a motion velocity of the micro/nano robot could be further calculated.

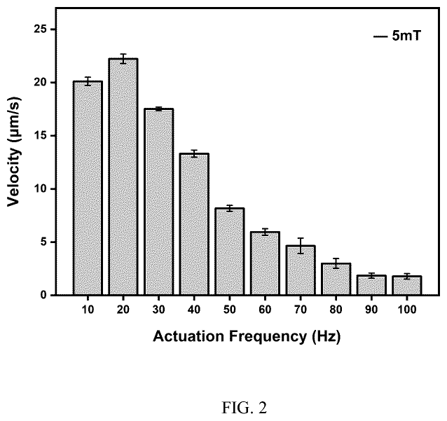

FIG. 2 shows motion velocity analysis results of the magnetite-based micro/nanorobot prepared in Example 1 of the present disclosure.

It can be seen from this figure that a motion velocity of the micro/nanorobot varies with a frequency of a magnetic field, and the micro/nanorobot has a maximum motion velocity of 22.22±0.45 μm/s at f=20 Hz, indicating that the magnetite-based micro/nanorobot has a better motion effect than the traditional passive transport mode.

### Cytotoxicity Experiment

Mouse mononuclear macrophages (RAW264.7, China Center for Type Culture Collection (CCTCC) at Wuhan University) were adopted as an object. Human venous endothelial cells were cultivated with a DMEM medium including 10% of fetal bovine serum and 1% of penicillin-streptomycin. The RAW264.7 cells were cultivated in a 37° C. and 5% CO₂ sterile environment, where the original medium was replaced with a fresh medium every two days until an appropriate cell confluency was reached. The adherent RAW264.7 cells were scraped off with a cell scraper and prepared into a cell suspension. 100 μL of the cell suspension with a cell concentration of 1×10⁵ cells/mL was inoculated into each well of a 96-well plate and cultivated for 12 h. The material was added at concentrations of 5 μg/mL, 10 μg/mL, 20 μg/mL, 40 μg/mL, 80 μg/mL, 160 μg/mL, 320 μg/mL, 640 μg/mL, and 1,280 μg/mL to the 96-well plate, and the 96-well plate was incubated for 12 h. The original medium was removed, and 100 μL of a mixed solution of CCK8 and a medium (volume ratio: 1:9) was added to each well. Then cells were cultivated for 1 h, and the absorbance was measured with a microplate reader (450 nm). Three parallel experiments were set, and a group without the material was set as a blank control group. Cell viability test results at different material concentrations were compared.

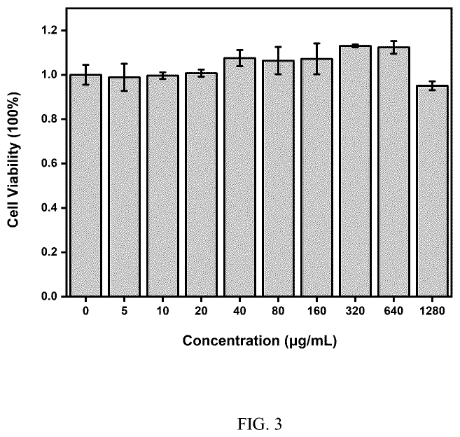

FIG. 3 shows cell viability test results of the magnetite-based micro/nanorobot in Example 1 of the present disclosure.

A cell viability of the blank control group is 100.00±4.50%, and cell viabilities of micro/nanorobot groups at different concentrations all are greater than 80%, indicating that the magnetite-based micro/nanorobot has excellent biocompatibility.

### DRUG RELEASE EXPERIMENT

In order to objectively evaluate a drug release behavior of the magnetite-based micro/nanorobot, an in vitro drug release experiment was conducted. An artificial gastric juice was prepared as follows: 2.0 g of sodium chloride and 3.2 g of pepsin were taken and added to 7.0 mL of concentrated hydrochloric acid, and deionized water was added to 1,000 mL to obtain an initial artificial gastric juice. A pH of the initial artificial gastric juice was adjusted with a 0.2 mol/L HCl solution to 1.2 to obtain the artificial gastric juice. An artificial intestinal juice was prepared as follows: 6.8 g of potassium dihydrogen phosphate was taken and dissolved in 500 mL of deionized water to obtain a potassium dihydrogen phosphate solution. 10 g of trypsin was taken and dissolved with an appropriate amount of water to obtain a trypsin solution. The potassium dihydrogen phosphate solution and the trypsin solution were mixed to obtain a mixed solution, and the mixed solution was diluted with water to 1,000 mL and adjusted with a 0.2 mol/L NaOH solution or a 0.2 mol/L HCl solution to a pH of 6.8 to obtain the artificial intestinal juice. 2 mg of the magnetite-based micro/nanorobot was dispersed in 10 mL of each of the artificial gastric juice and the artificial intestinal juice. The artificial gastric juice and the artificial intestinal juice with the magnetite-based micro/nanorobot dispersed each were placed in a constant-temperature shaker and slowly shaken at 37° C. and 100 rpm. At different time points, 1 mL of a sample solution was collected and centrifuged to obtain a supernatant, and an equal volume of the artificial gastric juice or the artificial intestinal fluid was supplemented. The supernatant was taken and tested by an ultraviolet spectrophotometer for absorbance at 305 nm. Cumulative drug release amounts and cumulative drug release rates at different time points were calculated based on a standard curve.

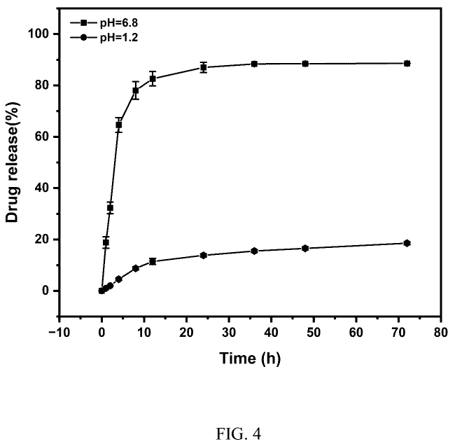

FIG. 4 shows drug release curves of the magnetite-based micro/nanorobot in Example 1 of the present disclosure under different pH values.

It can be seen from this figure that the magnetite-based micro/nanorobot has a very low drug release rate in the gastric juice with a pH of 1.2, but has a drug release rate of 88% in the intestinal juice with a pH of 6.8, indicating that the magnetite-based micro/nanorobot has high bioavailability for resveratrol and is a desired carrier for resveratrol drugs.

### Magnetocaloric Performance Experiment

2 mg of the magnetite-based micro/nanorobot was weighed and added to a 1.5 mL centrifuge tube, and the centrifuge tube was placed at a center position of a coil of a magnetocaloric instrument. Alternating magnetic fields with currents of 3.9 A, 4.2 A, and 4.5 A respectively were applied to the magnetite-based micro/nanorobot. At 0 min to 5 min, a temperature of the magnetite-based micro/nanorobot in the centrifuge tube was recorded every minute to obtain a change curve of the temperature over time.

Then, an alternating magnetic field with a current of 4.2 A was applied at 0 min to 5 min, and the application of the magnetic field was stopped at 5 min to 10 min, so as to allow a heating-cooling cycle. A temperature was recorded every 30 s. After 4 heating-cooling cycles, a magnetocaloric curve of the magnetite-based micro/nanorobot was obtained.

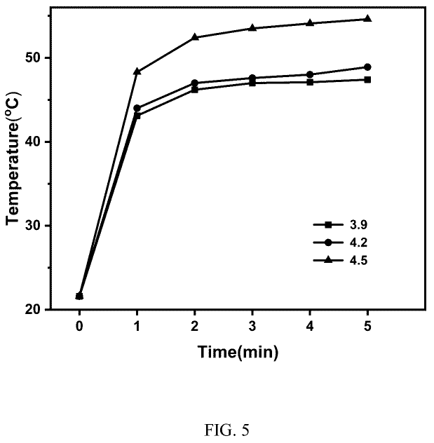

FIG. 5 shows temperature change curves of the magnetite-based micro/nanorobot prepared in Example 1 under actions of alternating magnetic fields.

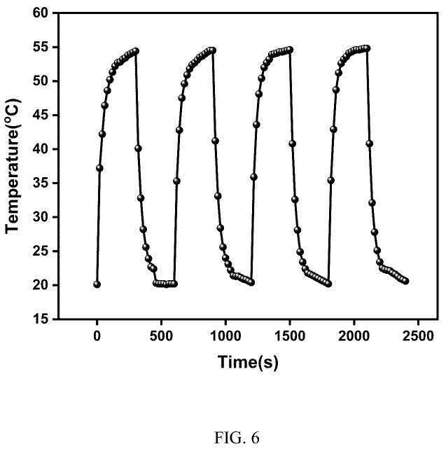

FIG. 6 shows a magnetocaloric curve of the magnetite-based micro/nanorobot prepared in Example 1 under an action of an alternating magnetic field.

It can be seen from FIG. 5 and FIG. 6 that the magnetite-based micro/nanorobot can be heated up to about 50° C. within 5 min under an action of an alternating magnetic field, and in a heating process of multiple cycles, the heating of the magnetite-based micro/nanorobot is stable. The above results indicate that the magnetite-based micro/nanorobot has excellent and stable magnetocaloric performance and a potential of magnetocaloric therapy, and can accelerate the recovery of an inflammatory disease.

### Oxidation Resistance Experiment

2,2′-Azino-bis(3-ethylbenzthiazoline-6-sulfonic acid) (ABTS) is a common reagent for an antioxidant experiment. An antioxidant activity of a material can be evaluated by measuring a reaction of ABTS with free radicals. An ABTS powder was weighed, dissolved with 80% ethanol, and diluted to obtain a 7 mM ABTS stock solution. The 7 mM ABTS stock solution and a 2.45 mM hydrogen peroxide solution were taken in a same volume, thoroughly mixed, and allowed to stand at room temperature in the dark for 1 h to obtain an ABTS working solution. 10 mg of the material was weighed and thoroughly mixed with 2 mL of the ABTS working solution to obtain a mixed solution, and the mixed solution was allowed to stand at room temperature in the dark for 6 min and centrifuged three times at 8,000 rpm to obtain a supernatant. An OD value of the supernatant was measured. An antioxidant activity of a sample was evaluated according to an experimental result.

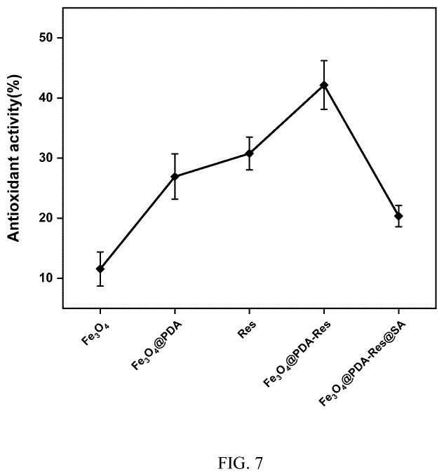

FIG. 7 is a schematic diagram of oxidation resistance of the magnetite-based micro/nanorobot prepared in Example 1 and components thereof.

It can be seen from this figure that the magnetite-based micro/nanorobot and the components thereof have oxidation resistance. According to a specific application scenario of the micro/nanorobot, a sodium alginate shell is dissolved at an enteritis site, which significantly improves an antioxidant activity of the magnetite-based micro/nanorobot at an inflammation site and allows the effective treatment of an inflammation.

What is not mentioned above can be acquired in the prior art.

Although some specific embodiments of the present disclosure have been described in detail by way of examples, those skilled in the art will appreciate that the above examples are provided for illustration only and not for limiting the scope of the present disclosure. A person skilled in the art can make various modifications or supplements to the specific embodiments described or replace them in a similar manner, but it may not depart from the direction of the present disclosure or the scope defined by the appended claims. Those skilled in the art should understand that any modification, equivalent replacement, and improvement made to the above embodiments according to the technical essence of the present disclosure shall be included in the protection scope of the present disclosure.

What is claimed is:

1. A magnetite-based micro/nanorobot, wherein the magnetite-based micro/nanorobot uses a polydopamine-coated magnetite as a carrier that is loaded with an anti-inflammatory drug and is coated with sodium alginate.

2. The magnetite-based micro/nanorobot according to claim 1, wherein the anti-inflammatory drug comprises one or more selected from a group consisting of resveratrol, curcumin, and sulfamethoxazole.

3. The magnetite-based micro/nanorobot according to claim 1, wherein the polydopamine-coated magnetite is produced through polymerization of dopamine hydrochloride on a surface of a magnetite under an alkaline condition.

4. The magnetite-based micro/nanorobot according to claim 2, wherein the resveratrol is adsorbed on a surface of the polydopamine-coated magnetite through an electrostatic interaction.

5. The magnetite-based micro/nano robot according to claim 3, wherein the magnetite is spherical and has a nano-scale size; and/or, the magnetite-based micro/nanorobot is spherical and has a nano-scale size.

6. A preparation method of the magnetite-based micro/nanorobot according to claim 1, comprising the following steps:
S1, preparing the magnetite through hydrothermal synthesis;
S2, polymerizing dopamine hydrochloride on a surface of the magnetite under the alkaline condition to obtain the carrier;
S3, dissolving and adding the anti-inflammatory drug of resveratrol to the carrier, shaking in dark for a period of time, washing, and lyophilizing to obtain a magnetite complex loaded with the resveratrol; and
S4, coating the sodium alginate on a surface of the magnetite complex loaded with the resveratrol through a water-in-oil emulsion process to obtain the magnetite-based micro/nano robot.

7. The preparation method according to claim 6, wherein in the S3, the anti-inflammatory drug is the resveratrol, and the resveratrol is added to methanol and shaken in the dark for thorough dissolution; and a concentration of the resveratrol dissolved is 2.5 mg/mL to 7.5 mg/mL.

8. The preparation method according to claim 6, wherein in the S3, a mass ratio of the carrier to the resveratrol is 1:(5-15).

9. The preparation method according to claim 6, wherein the step S4 comprises:
adding the sodium alginate and the magnetite complex loaded with the resveratrol to deionized water, and mechanically stirring for thorough dissolution to obtain an aqueous phase; mixing liquid paraffin, Span-80, and Tween-80, stirring until clear, and cooling to obtain an oil phase; and adding the oil phase to the aqueous phase.
